(19) United States
(12) Patent Application Publication
(10) Pub. No.: US 2026/0306828 A1
(43) Pub. Date: Oct. 8, 2026

(54) INDUCING AND MONITORING MUSCLE GROWTH AND ACTIVITY IN A WEARABLE NEUROPROSTHESIS

(71) Applicant: Philip Muccio, Ann Arbor, MI (US)

(72) Inventor: Philip Muccio, Ann Arbor, MI (US)

(21) Appl. No.: 19/108,155

(22) PCT Filed: Sep. 4, 2023
(86) PCT No.: PCT/US2023/031948

§ 371 Date: Mar. 1, 2025

Related U.S. Application Data
(60) Provisional application No. 63/374,588, filed on Sep. 5, 2022.

(51) Int. Cl. A61N 1/04; A61B 5/397; A61N 1/36

(52) U.S. Cl. CPC A61N 1/0452; A61B 5/397; A61N 1/0476; A61N 1/0484; A61N 1/36031; A61N 1/36034

(57) ABSTRACT

An electrical stimulator device, comprising a first pair of electrodes configured to be applied to a first target muscle group of a user; at least a second pair of electrodes configured to applied to a second target muscle group; a reference electrode configured to be applied to a location on the body of the user; a control system having switch circuitry; a stimulation device; and an EMG measurement device. The control system switch circuitry operatively connects the stimulation device and the EMG measurement device to the first pair of electrodes and to the reference electrode such that when the stimulation device is activated, the first electrode pair is operative to stimulate the first muscle group. A method of operating a wearable stimulation device is also disclosed.

### RELATED APPLICATION

This application is a national phase of and claims priority to International Patent Application No. PCT/US23/31948, filed Sep. 4, 2023, which claims priority to US Provisional Application No. 63/374,588, filed Sep. 5, 2022, the contents of each of which are incorporated herein by reference in their entireties.

### TECHNICAL FIELD

The present disclosure relates generally to wearable therapy devices, and more specifically to a wearable therapy device that provides muscle stimulation and takes electromyography (EMG) measurements of the muscles to allow for tracking the efficacy of the device.

### BACKGROUND

Muscle paralysis can lead to a host of deleterious health effects that diminish quality of life, reduce skeletal function, and impair physiological processes. Patients who succumb to neurological injury or disease affecting the brain or spinal cord will spend a lifetime fighting atrophy, spasticity, joint stiffness and contracture, pain, as well as diminished or absent arm, trunk, and leg function. These problems can disrupt vocation and life pursuits well beyond simple injuries that heal. Spinal cord injury, for example, can paralyze all four extremities and incapacitate an individual for life, forcing those individuals to ambulate in a wheelchair, breathe on a ventilator, or even live separate from family due to the extensive need for medical care.

Paralysis of a lesser degree that affects one or two muscles, can still be very disabling. For example, paralysis of the tibialis anterior and extensor digitorum longus will cause foot drop and in this condition, the foot drags or causes the patient to trip.

Paralysis can generally be divided into two forms: 1) complete paralysis, and 2) incomplete paralysis. Complete paralysis is the total loss of brain connectivity to a group of muscles. For instance, one can lose total brain control to an extremity wherein there is no elicitation of muscle contraction possible when the patient attempts to move the limb. There is a total block of neurological signaling from the brain to all muscles in that extremity. Incomplete paralysis is the partial loss of brain control to the affected extremity. In this scenario, some brain signals reach the affected muscles, but the signals are compromised and although the muscles will contract, they cannot contract in full force.

In both complete and incomplete paralysis there is a reduction in muscle strength due to the reduction of brain-initiated signals and the loss of muscle activity. This is called atrophy and it weakens muscles. Without daily movement and exercise, atrophy can become a significant problem in the paralyzed that could lead to problems such as inability or decreased ability to move limbs or stabilize the trunk, diminished circulation to limbs, an increase in susceptibility to pressure sores and slow wound healing, an increase in susceptibility to diabetes, an onset of chronic back pain or pain in various joints and certainly a loss of independence and control over one's vocational pursuits.

Atrophy is not the only problem to plague the paralyzed. Muscle spasms are erratic, involuntary muscle contractions that can stiffen joints, restrict movement, and force limbs into unwanted positions. Spasms can occur in one or more muscles, but they typically affect the flexor side of a joint and that usually causes the joint to flex and stay flexed. When joints stay flexed for prolonged periods of time the spastic muscle(s) tend to shorten and stiffen to such an extent that extension of the joint becomes much more difficult or even impossible. A typical example of this is when spasms present in the biceps muscle. Biceps are responsible for flexing the elbow. When the biceps become spastic, the elbow will flex and stay flexed. This leads to stiffness and contracture at the elbow that then diminishes arm function since it becomes very difficult to straighten the arm against the powerful spastic biceps.

The combined effects of atrophy, spasms and reduced cortical control (from brain or spinal cord injury or disorder) on the body is what manifests in partial or complete paralysis and this is what leads to physical disability in most patients suffering from neurological disorders.

Neuromuscular electrical stimulation (NMES) is a method of artificially contracting muscles when paralysis prevents or diminishes voluntary muscle contractions from taking place. NMES can be useful for stopping or at least slowing the progression of muscle atrophy. Indeed, NMES may actually induce hypertrophy under certain conditions.

To understand a muscle's response to electrical stimulation, surface electromyography (sEMG) is employed to provide an indirect indication of the strength of a muscle contraction. Typical sEMG arrangements include an array of three cutaneious electrodes; a pair of electrodes are attached to a substrate that may be worn by a patient, with the third electrode being used as a reference electrode or ground. During a measurement event, voltage pulses are applied across the pair of electrodes and an electric field generated by the collection of the muscle fibers as they contract are amplified to provide an EMG measurement, i.e., the electrical output of a group of motor units. There are known systems that utilize the same pair of electrodes to both apply stimulation, as well as measure the EMG impulses of the same muscle to which the stimulation is applied.

However, if the same electrode pair is used for both events, different time sections need to be allocated for different activities. For example, a known periodic scheme 10, as shown in FIG. 1, includes sending positive and negative stimulation pulses 12, 14, waiting and measuring the EMG 16, and then waiting again, and sending the next positive and negative stimulation pulses again to repeat the process. Accordingly, the time for application of therapy is extended to accommodate the time lag caused by the needed wait for measuring the EMG. What is needed is a measurement scheme that reduces therapy time.

The strength of an EMG signal is proportional to the number of contracting motor units. As the number of contracting motor units increases, the voltage and electric field generated also increase. Correspondingly, as a muscle hypertrophies, each fiber of the muscle generates a stronger voltage output and thus this also contributes to an increase in EMG signal intensity. As such, an EMG signal provides an indirect measure of whether a muscle is increasing in strength over time, staying the same, or declining.

Inertial movement units (“IMUs”) are electronic devices that measure and report a body's specific force, position, angular rate, velocity, and acceleration relative to a given sensor on the body. IMUs are useful in determining the movements of a given body part when attached to a such body part.

To enable consistent stimulation to each targeted area, as well as to record EMG and/or IMU measurements, both the placement of the electrodes for stimulation, EMG measurement, as well as placement of IMUs must be at the same location each time to enable monitoring of the therapy and the effectiveness of such therapy. Accordingly, what is needed is a way to ensure proper placement of the electrodes and/or the IMUs.

### SUMMARY

In accordance with a first aspect of the disclosure, an electrical stimulator device is disclosed. The electrical stimulator device comprises a first pair of electrodes configured to be applied to a first target muscle group of a user; at least a second pair of electrodes configured and applied to a second target muscle group of the user; and a reference electrode applied to another location on a body of the user. A control system having switch circuitry is also provided, as well as a stimulation device and an EMG measurement device. The control system switch circuity operatively connects the stimulation device and the EMG measurement device to the first pair of electrodes and to the reference electrode such that when the stimulation device is activated, the first electrode pair is operative to stimulate the first muscle group.

The switch circuity is configured to turn the stimulation device off from the first electrode pair and turn the stimulation device on for the second electrode pair. In addition, the switch circuity may be configured to turn the stimulation device off for all electrode pairs and turn on the EMG measurement device for one of the first and second electrode pair, and the reference electrode. The electrical stimulator device may have more than two electrode pairs.

In accordance with one aspect of the disclosure, an input device is operatively connected to the control system. The input device may be a handheld device that is fixed to the electrical stimulator by a wired connection. Alternatively, the input device may be a mobile device, a tablet, or computer that is wirelessly connected to the electrical stimulator.

In accordance with another aspect of the disclosure, a server is operatively connected to the control system, the server having a memory to store EMG measurements and provide computational analysis of the EMG measurements to allow for a determination of adjustment of therapy parameters.

In accordance with another aspect of the disclosure, one or more inertial movement units are operatively connected to the control system, wherein the inertial movement units are configured to track movement and position of one or more muscle groups. More specifically, data corresponding to different locational positions of such muscle groups are transmitted to the control system and/or the server to provide computational analysis of the position data and allow for a determination of adjustment of therapy parameters.

In accordance with another aspect of the disclosure, the electrical stimulator device is a wearable garment. For example, to apply stimulation therapy to the quadriceps and hamstring muscles, the wearable garment may be a pair of compression shorts. For applying therapy to an arm, the wearable garment may be a compression sleeve. There are significant advantages of having a wearable garment for applying stimulation. For example, therapy can be applied at an individual's home and on a schedule that is convenient for the individual. In addition, the electrodes can be accurately placed every time, as the garment will provide repeatability in the positioning of the electrodes.

In accordance with another aspect of the disclosure, a method of applying a stimulation therapy is also disclosed. The method begins by positioning an electrical stimulator device such that a first set of electrodes are positioned across a first target muscle group of a user, a second set of electrodes are positioned across a second target muscle group of the user; and a reference electrode is positioned on another location on the body of the user. One or more stimulation parameters for a stimulation cycle are set and the stimulation cycle for the first muscle group is initiated by providing a biphasic pulse followed by an EMG measurement collection and repeating application of the biphasic pulse followed by the EMG measurement for a predetermined time period. Data from the EMG measurement collection is aggregated to demonstrate a response to the stimulation pulses by the first muscle group.

In accordance with another aspect of the disclosure, after the stimulation cycle is performed for the first muscle group, a second stimulation cycle is performed for the second muscle group, by providing a biphasic pulse followed by EMG measurement collection for a second predetermined time period, and aggregating data from the EMG measurement collection for the second predetermined time period to demonstrate a response to the stimulation pulses by the second muscle group.

In accordance with another aspect of the disclosure, voluntary contractions of the first muscle group may be initiated and EMG measurements of the voluntary contractions for a predetermined number of times are taken and recorded for the voluntary contractions. The EMG measurements of the voluntary contractions are compared with the EMG measurements collected after stimulation to make a determination on the progress of therapy or making any adjustments to the therapy.

In accordance with a further aspect of the disclosure, positional data of the first muscle group may be tracked over a predetermined time period. The positional data may be used to determine how a muscle group is responding to therapy.

In accordance with another aspect of the disclosure, after the stimulation cycle is applied to the first muscle group, a second stimulation cycle is initiated for the second muscle group by providing a biphasic pulse followed by an EMG measurement collection and repeating application of the biphasic pulse followed by the EMG measurement for a second predetermined time period. After the stimulation cycle is initiated for the first muscle group, a second stimulation cycle is initiated for the second muscle group, and an EMG measurement collection is performed during a common gap between each biphasic pulse of each stimulation cycle.

In accordance with another aspect of the disclosure, a third electrode pair is positioned across a third muscle group. A stimulation cycle is initiated for each of the first, second and third muscle groups. The stimulation cycle for the second muscle group is delayed until after more than one biphasic pulses are applied to the first muscle group, and the stimulation cycle for the third muscle group is delayed until after the more than one biphasic pulses are applied to the second muscle group. An EMG measurement collection is performed during a common gap between each biphasic pulse of the biphasic pulses of the first, second and third muscle groups.

### BRIEF DESCRIPTION OF THE DRAWINGS

The foregoing and other features and advantages of the present disclosure will become apparent to those skilled in the art to which the present disclosure relates upon reading the following description with reference to the accompanying drawings, in which:

FIG. 1 is a prior art EMG measurement scheme for a single channel stimulation arrangement;

FIG. 2 is an exemplary arrangement of a multiple channel stimulation and EMG measurement arrangement in accordance with the present disclosure;

FIG. 3 is a measurement scheme for a multi-channel stimulation arrangement illustrating possible interference;

FIG. 4 is a system diagram for a stimulation system employing the stimulation and EMG measurement arrangement of FIG. 2;

FIG. 5 is a simplified circuit diagram for an electrode arrangement for stimulation and EMG measurement;

FIG. 6A is a segment of a stimulation and EMG measurement scheme for a pair of muscle groups;

FIG. 6B is an alternative stimulation and EMG measurement scheme for a pair of muscle groups;

FIG. 7 is a multi-segment measurement scheme for a multi-channel stimulation and EMG measurement arrangement with a timing shift in accordance with the present disclosure;

FIG. 8 is a multi-segment of a synchronized measurement scheme for a multi-channel stimulation and EMG measurement arrangement in accordance with the present disclosure;

FIG. 9 is a multi-segment of a measurement scheme for a multi-channel stimulation and EMG measurement arrangement with a pulse elimination in accordance with the present disclosure;

FIGS. 10A-10C are representative Peak EMG Signals taken over progressive months of a targeted muscle group;

FIGS. 11A-11C are average Peak EMG trend lines over progressive months showing possible outcomes related to muscle growth;

FIGS. 12A and 12B are front and rear views of a user that is undergoing an exemplary stimulation operation to a first leg muscle;

FIGS. 12C and 12D are front and rear views of a user that is undergoing an exemplary stimulation operation to a second leg muscle;

FIGS. 13A and 13B are front and rear view of a user that is undergoing an exemplary stimulation operation to a third leg muscle;

FIGS. 13C and 13D are front and rear views of a user that is undergoing an exemplary operation to a fourth leg muscle;

FIG. 14 is a flowchart demonstrating an exemplary method of operating a wearable stimulation and EMG measurement arrangement on a completely paralyzed individual;

FIG. 15 is a flowchart demonstrating an exemplary method of operating a wearable stimulation and EMG measurement arrangement on a partially paralyzed individual;

FIG. 16 is an exemplary chart illustrating three months of data utilizing the method of FIG. 14;

FIG. 17 is an exemplary chart illustrating three months of data utilizing the method of FIG. 15;

FIG. 18 is a front view of an individual with hemiplegia, illustrating potential locations for inertial movement units to track arm movement;

FIG. 19 is a side view of an individual wearing a sleeve that incorporates a stimulation and EMG measurement arrangement, as well as having inertial movement units;

FIGS. 20A-20C illustrate three months of positional data after the application of therapy utilizing the sleeve of FIG. 19; and

FIGS. 21A-21C illustrate a user wearing the sleeve of FIG. 19 in a variety of different positions.

### DETAILED DESCRIPTION

Referring now to the discussion that follows, and to the drawings, illustrative approaches to the disclosed systems and methods are shown in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

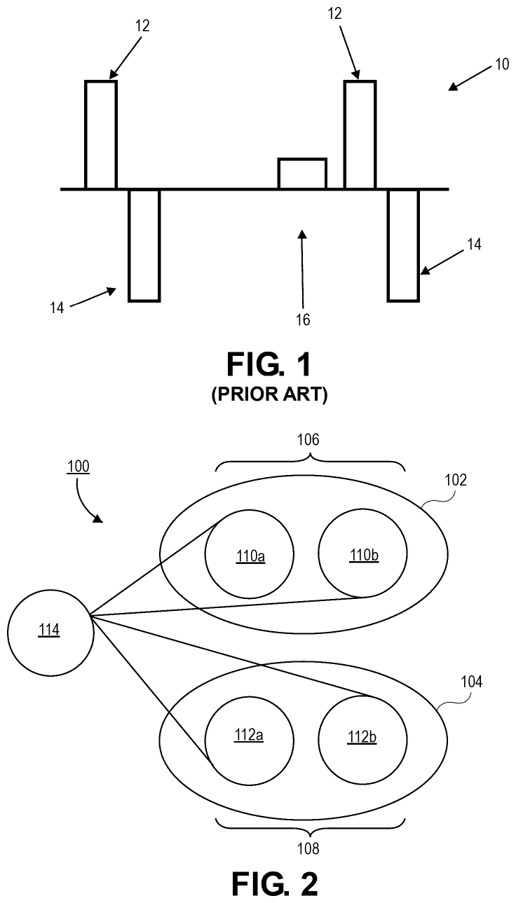

Referring to FIGS. 1-11C, various arrangements of multiple channel stimulation and EMG measurement arrangements are illustrated, all of which can be utilized for people having a muscle or muscles that are partially paralyzed, a muscle or muscles that are completely paralyzed, or those needing other pain relief, spasm relief, and the like. For clarity, muscles that have been partially paralyzed, will have a reduced EMG intensity signal compared to a muscle that has not been paralyzed (i.e., full voluntary control will exist in individuals who can impart full brain control over the muscle) due to a reduction in the quantity of motor units that are recruited as well as due to disuse atrophy. The signal output strength will depend on the degree of paralysis and to what extent atrophy has taken place.

In contrast, a muscle that is completely paralyzed cannot generate its own EMG signal upon command as the muscle is completely cut off from the brain. In this instance, any contraction must be induced by electrical stimulation.

Referring to FIG. 2, in instances where it is desired to apply stimulation, an exemplary stimulation and measurement arrangement 100 is disclosed herein. The stimulation and measurement arrangement 100 allows for multiple muscle groups 102, 104 to be stimulated. In one exemplary arrangement, the stimulation system 100 includes at least first and second electrode pairs 106, 108. Each electrode pair 106, 108, includes first and second electrodes 110, 112. While only two electrode pairs 106, 108 are shown, it is understood that the stimulation system 100 may include more than two electrode pairs 106, 108. In addition, the stimulation system 100 may also include only a single electrode pair 106. Each electrode pair 106, 108 is configured to be attached to an area of interest, i.e., a muscle group, on a wearer.

In prior art systems, each electrode pair is attached to its own reference electrode that is used to measure a value of a potential difference between the electrodes in a pair of electrodes based on a reference value of the reference electrode. The reference electrode acts as a ground and thus must be placed away from the EMG detecting surfaces, i.e., on an electrically neutral surface on a body of the user. However, in prior art systems, supplying separate reference electrodes for each electrode pair necessarily increases the number of electrodes needed.

In accordance with an aspect of the disclosure, each of the electrodes 110a, 110b, 112a, 112b may each be connected to a common reference electrode 114. In the instance arrangement, both electrode pairs 106, 108 share the same reference electrode 114, as will be explained in further detail below. In this manner, the number of needed electrodes may be significantly reduced, thereby reducing complexity of an arrangement, as well as reducing cost associated with same.

In some prior art systems, a different electrode is used for measuring EMG than the electrodes used for stimulation. However, with such arrangements, due to the different locations, the EMG measuring electrodes are actually measuring a different muscle group than the muscle group being stimulated. In contrast, the arrangement disclosed herein uses the same electrode pair 106 for stimulation and EMG measurement, thereby allowing for measurement of the EMG signal of the same muscle group after stimulation.

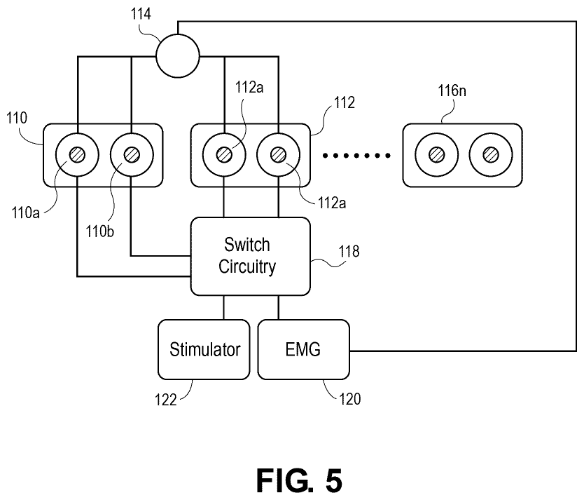

Referring to FIG. 5, to maximize use of the electrode pairs 106, 108, (including 116n representing any additional electrode pairs), in one exemplary arrangement, switch circuitry 118 is operatively connected to each of the electrode pairs 106, 108, . . . 116n, as well as the reference electrode 114. In addition, the switch circuitry 118 is also operatively connected to an EMG measurement device 120 and a stimulator device 122. With this arrangement, the number of reference electrodes may be minimized for taking EMG measurements.

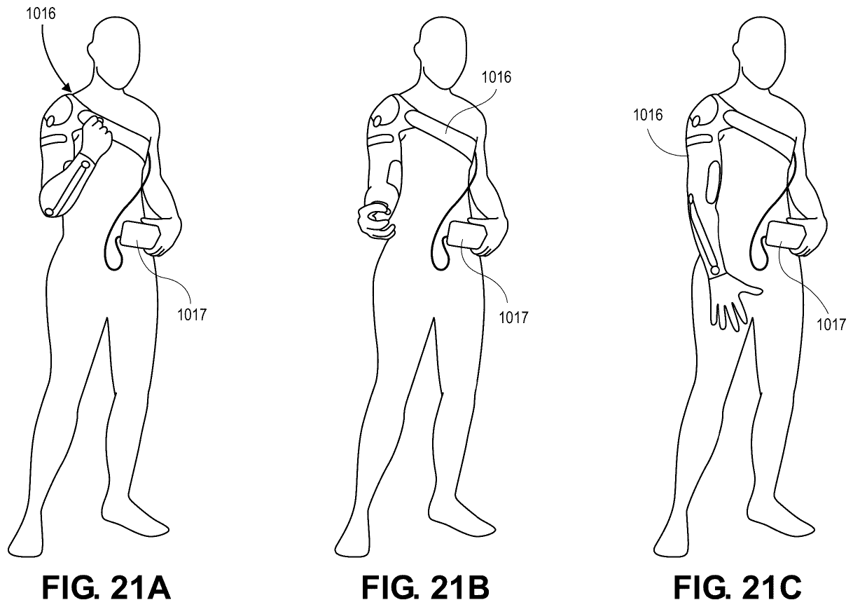

Referring to FIG. 4, an exemplary electrical stimulation system 200 that utilizes the stimulation and measurement arrangement 100 is illustrated. The stimulation system 200 includes an input device 202 that is operably connected to a control system 204 to operate the stimulation and measurement arrangement 100. In one exemplary arrangement, the input device 202 may be a remote device, such as a mobile phone or a tablet, that is operatively connected to the control system 204. Alternatively, the input device 202 may be a handheld device hardwired to the control system 204, such as that shown in FIGS. 21A-21C.

The control system 204 includes a control algorithm to employ an appropriate signal pattern to different electrode pairs 106, 108, 116n that are applied to predetermined muscle groups 102, 104. In one exemplary arrangement, the control system 204 incorporates the switch circuitry 118, as well as the stimulator 122 and EMG measurement device 120.

The control system 204 is connected to the electrode pairs 110, 112 and 116n, as well as the reference electrode 114. As discussed above, the electrode pairs 110, 112, 116n, and the reference electrode 114 are to be applied to different muscle groups 102, 104. In one exemplary arrangement, the electrode pairs 110, 112, 116n may be incorporated into a wearable garment 206, for example, a pair of form fitting shorts that conform to a person's thigh, or an arm sleeve that confirms to a person's shoulder and arm, as will be described in further detail below.

A server 208 is operatively connected to the control system 204 and serves to collect, and/or analyze data received from the electrode pairs 110, 112, 116n. The server 208 may be operatively configured to deliver the data to a display device (not shown), to allow a practitioner to extrapolate the data to monitor the efficacy of the stimulation and measurement arrangement 100, as well as to use the data to make changes to a treatment plan being employed by the stimulation and measurement arrangement 100.

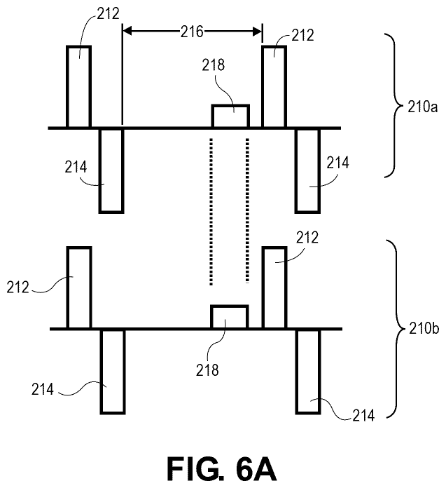

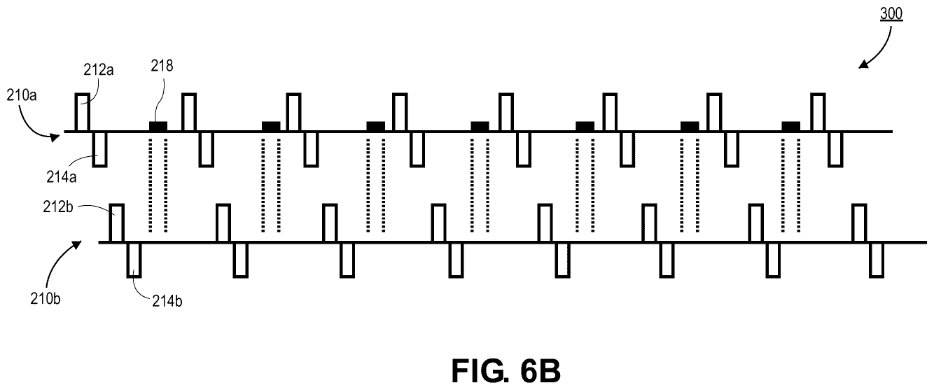

The switch circuitry 118 includes stimulator switches (not shown) and EMG switches (not shown). During operation of the stimulator and measurement arrangement 100, a control signal from the control system 204 will activate stimulator switches to provide biphasic pulses to the first and second muscle groups 102 and 104. Referring to FIG. 6A, a segment 210a, 210b of a stimulation and EMG measurement activity for each muscle group 102, 104, respectively is shown. After application of an initial stimulation, i.e., application of positive and negative pulse segments 212, 214 to each of the first and second muscle groups 102, 104, a timing device (no shown) operatively connected to the control system 204 controls the application of each pulse segment 212, 214, as well as timing between stimulation pulses. Once the initial stimulation pulse segments 212, 214 are applied, a pause of predetermined length 216 is applied, until the next stimulation pulse operation is performed. The pause 216 is performed to allow for the excitation to propagate through the muscle fibers such that the next stimulation event does not inaccurately impact the muscle fiber response.

During the pause segment 216, EMG measurements 218 are taken. More specifically, the switch circuitry 118 of the control system 204 also includes EMG switches (not shown). After a stimulation pulse segment, the control system 204 triggers stimulation switches to an “off” position, and then toggles the EMG switches to turn on the EMG measurement function for the first and second electrode pairs 110, 112. This action allows sampling of the EMG measurements to be taken of the muscle groups 102, 104 between pulses. The collected EMG measurements may then be transmitted to the server 208, as will be discussed in greater detail below.

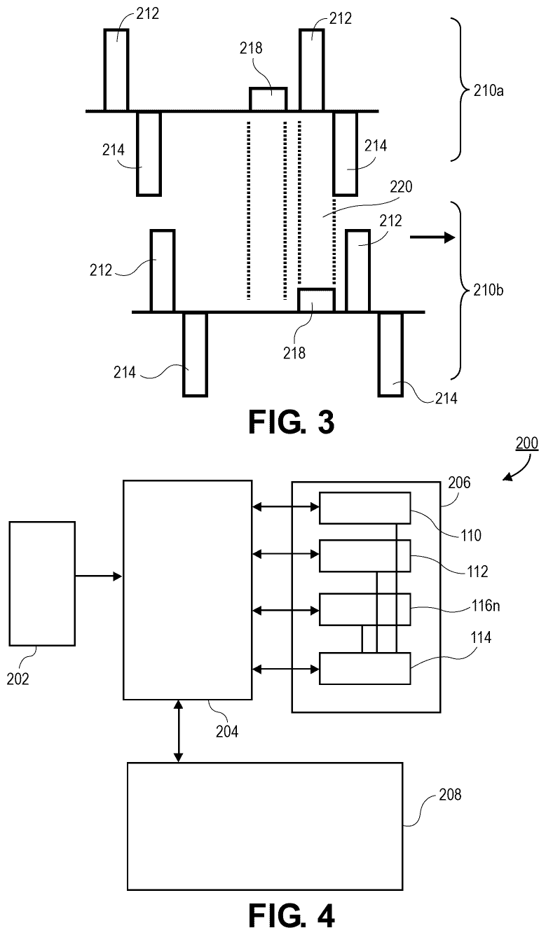

Referring to FIG. 3, when utilizing the stimulation and EMG measurement arrangement 100, i.e., stimulating and measuring two different muscle groups 102, 104, one issue that can occur is electrical interference, if the stimulation pulses 210 from the first muscle group 102 are active during an EMG measurement 218 of the second muscle group. This electrical interference will prevent an accurate EMG measurement 218 from being taken from the second muscle group 104.

Referring back to FIG. 6A, in one exemplary arrangement, each of the muscle groups 102, 104 may be subjected to synchronized stimulation and EMG measurement events. In this arrangement, the EMG measurements may take place for both muscle groups 102, 104 without interference.

Referring to FIG. 6B, a further exemplary arrangement of a stimulation and EMG measurement scheme 300 is shown. In the scheme 300, the EMG measurements are still taken between stimulation pulses 210a, 210b of two different muscle groups 102, 104. However, the muscle stimulations for each muscle group are out of phase with each other. For example, the first muscle group 102 is stimulated, with the pulse segments 212a, 214a, with the second muscle group 104 being stimulated immediately after the pulse segment 214a ends. After the pulse segments 212b, 214b for the second muscle group 104 are initiated, the EMG measurements 218 are taken for the muscle group 102 before the stimulation process is repeated.

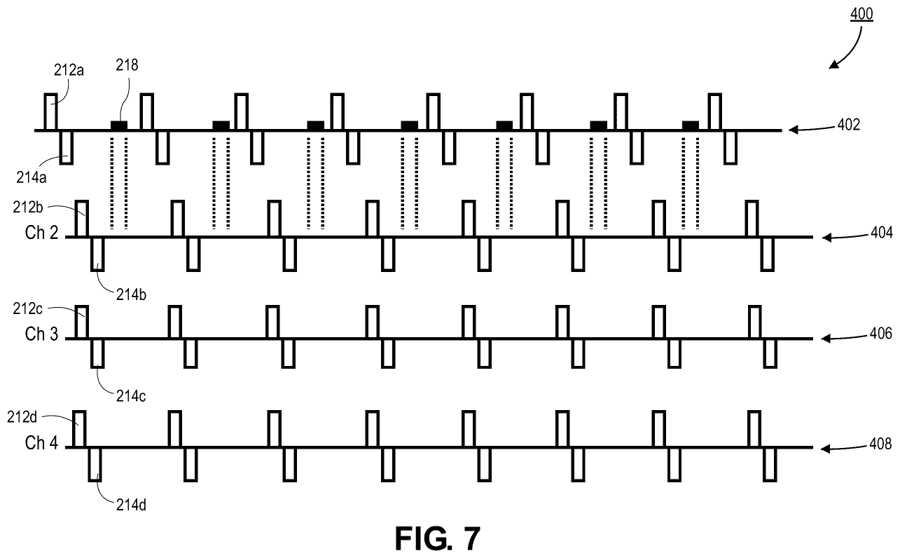

Referring to FIG. 7, another exemplary arrangement of a stimulation and EMG measurement scheme 400 is shown. In the scheme 400, the stimulation and EMG arrangement 100 is applied to four separate muscle groups, represented by stimulation pulse channels 402, 404, 406, and 408. In the arrangement shown, the first muscle group 102 is stimulated with stimulation pulses 212a, 214a, similar to that already shown and discussed above, as shown in stimulation pulse channel 402. Similarly, the second muscle group 104, and a third muscle group, and a fourth muscle group are also stimulated with stimulation pulses 212b-d, 214b-d, respectively, as shown in stimulation pulse channels 404, 406 and 408, respectively. In this arrangement, the first muscle group 102 is undergoing an EMG measurement 218, while the second through the fourth muscle channels are also being stimulated. With this arrangement, though the second through the fourth muscle channels have shifted to the right, there is still time between pulses to measure an EMG signal of the first muscle group 102, without electrical interference from the second through fourth muscle groups.

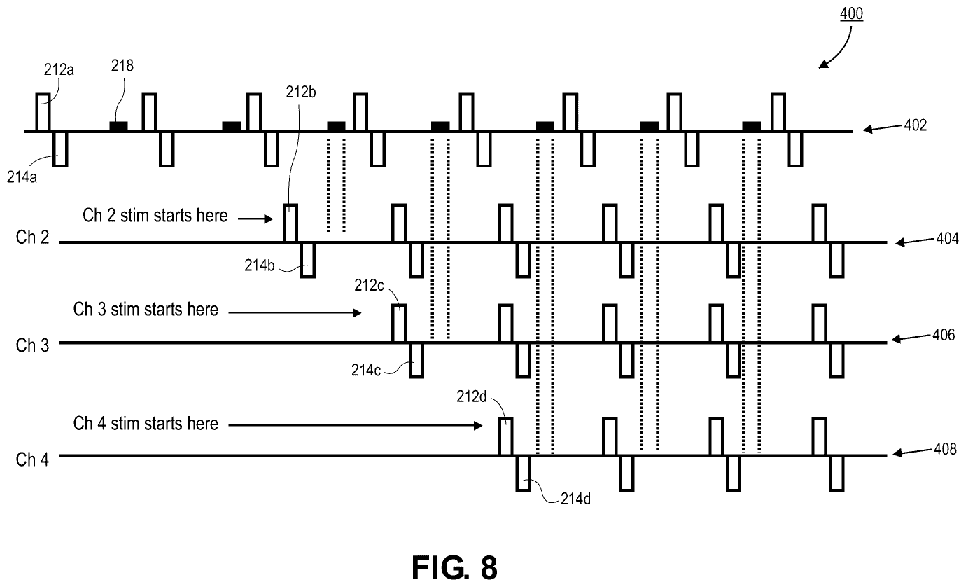

In one exemplary arrangement, referring to FIG. 8, after stimulation of the first muscle group 102 begins, the control system 204 is configured with a timing circuit that is synchronized to start stimulation of the second to fourth muscle groups, i.e., stimulation pulses 212b-d, 214b-d in a manner to allow for the EMG measurements 218 to be taken of the first muscle group 102. In one exemplary arrangement, the initiation of stimulation of the second to fourth muscle groups are staggered. Alternatively, the initiation of stimulation of the second to fourth muscle groups may be time to start simultaneously, as shown in FIG. 7.

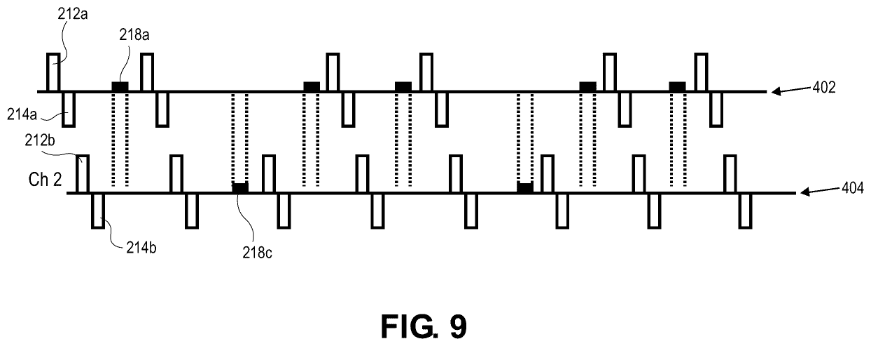

In one exemplary arrangement, referring to FIG. 9, rather than taking EMG measurements 218a, 218b one muscle group 102, 104 at a time, EMG measurements 218a, 218b, may be taken of both muscle groups while stimulation is applied to each of the muscle groups 102, 104. More specifically, after initial stimulation pulses 212a, 214a are applied to the first muscle group 102, as well as initial stimulation pulses 212b, 214b to the second muscle group 104, and an EMG measurement 218a is taken of the first muscle group 102. Because the stimulation pulses 212b, 214b of the second muscle group 104 are initiated so as to create a sufficient pause between stimulation pulses for the first muscle group 102 and the second muscle group 104, the EMG measurement 218a is not subjected to electrical interference. In addition, EMG measurements 218b of the second muscle group 104 may also be taken. To further guard against electrical interference, stimulation pulses from the second muscle group 104 are eliminated from the stimulation pulses from the first muscle group 102 during the period of time that the EMG measurement 218a is being taken.

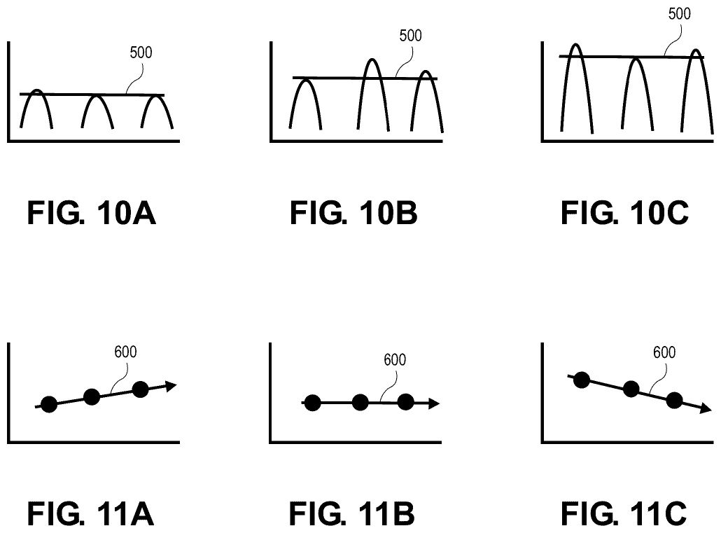

One of the advantages of the present arrangement of utilizing the stimulation and EMG measurement arrangement 100, is that EMG measurements may be tracked over predefined time periods which allow health practitioners to analyze the results of the treatment as a function of peak EMG signals in defined muscle groups. For example, when an individual exerts a maximum, voluntary contraction, the EMG will produce a peak value. In one exemplary method, a series of multiple maximum, volitional contractions may be collected and aggregated to calculate an average peak EMG signal. If measured over time, an EMG trend line 500 will indicate the status of the muscle being measured. For example, referring to FIGS. 10A-10C, the control system 204 may be configured to have peak EMG measurements taken of a muscle group 102 during therapy sessions over a predefined time period. The EMG measurements may be transmitted to the control system 204 and/or the server 208 and subjected to analysis to generate average peak EMG signals over the predetermined time period. The average peak EMG signals may be utilized to output a trend line, i.e., transmitted to a display device (not shown) to graphically illustrate the status of the muscle group. Such information will assist in determining the effectiveness of a stimulation treatment, as well as to track progress of the muscle group.

As an example, FIG. 10A represents peak EMG signals measured during the first month of stimulation therapy. The trend line 500 reaches a first peak EMG level during the first month. During the next month, as shown in FIG. 10B, additional measurements are taken, and the trend line 500 has reached a second, higher peak EMG level, indicating an increase of the average peak EMG signal. Finally, during a third month, as shown in FIG. 10C, the average peak EMG has generated a trend line 500 that is a higher level than the second month. When looking at all three months' data, the trend indicates that the average EMG peak level is increasing such that the therapy is producing beneficial results.

In addition to tracking average peak EMG levels over set predetermined time periods, this data may also be plotted together to make a determination, and hence drive therapy strategy decisions, as to the status of hypertrophy in a select muscle group. For example, as shown in FIG. 11A, each of the average peak EMG trend line 500 values from FIGS. 10A-10C are plotted together, to illustrate an increasing trend line 600. Should the average peak line 500 values from FIGS. 10A-10C, when plotted together remain generally horizontal, that would indicate that the muscle group is neither undergoing hypertrophy nor atrophy. This may indicate that the treatment is having no or little impact on the muscle group. Alternatively, this may indicate that the therapy is preventing muscle atrophy. In some patients, they lose muscle over time so preventing muscle atrophy would be a benefit). Finally, referring to FIG. 11C, if the average peak line 500 values from FIGS. 10A-10C, when plotted together is decreasing over time, the muscle group is undergoing atrophy. In this case it could be assumed that the therapy is not preventing the loss of muscle and therefore the protocol could be adjusted in terms of strength of the intensity of stimulus, increasing the amount of time the stimulus is applied per session or increasing the number of sessions per week. It could also mean that the patient is not using the system as prescribed, meaning the compliance report would show the patient using the system less than what is prescribed. The software would notify the patient that usage is less than prescribed and recommend using it as prescribed to prevent muscle atrophy.

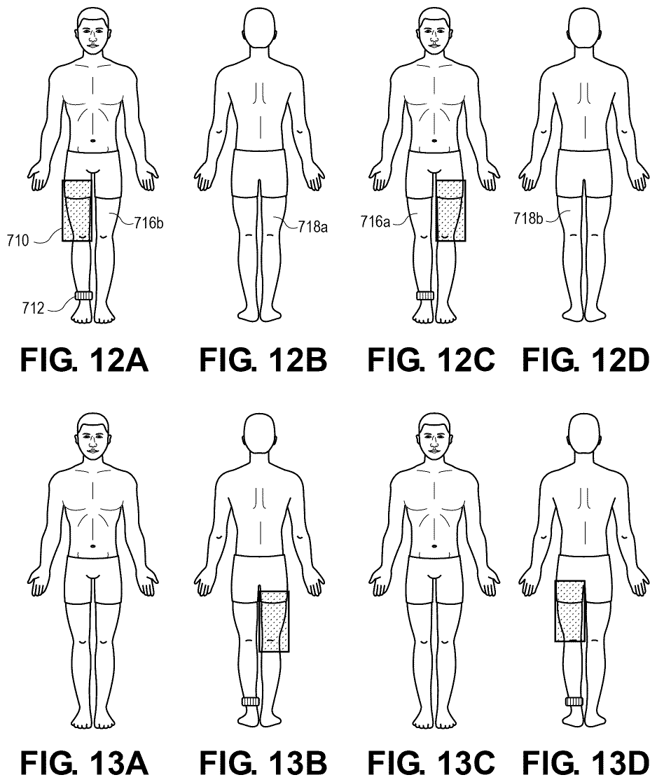

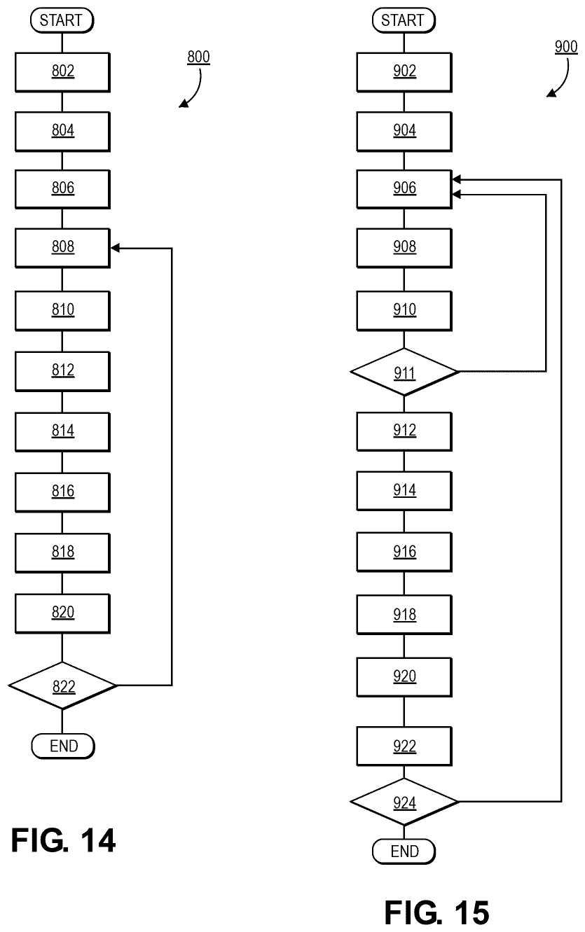

Referring to FIGS. 12A-13D, as well as FIG. 14, a method 800 of operating an exemplary configuration of how a wearable stimulation and EMG measurement arrangement 100 on a completely paralyzed individual 700 operates will now be described.

Referring to FIGS. 12A, 12C, 13B, 13D, and 14, the method 800 begins in a step 802 with providing a wearable device that incorporates the electrode pairs 106, 108, and reference electrode 114. In one exemplary arrangement, a wearable device may be a garment 710, such as a pair of shorts (a portion of which is schematically represented in these figures), or an arm sleeve 1016 (shown in FIGS. 21A-21C). As previously discussed, one of the challenges to delivering consistent therapy is ensuring consistent, and repeated proper placement of the electrode pairs 106, 108. To address this issue, electrode pairs 106 may be integrated into the wearable garments 710, 1016, such that when the garments are worn, the electrode pairs 106, 108 are operatively positioned on predetermined muscle groups. Because the position of the electrode pairs 106, 108 are fixed within the wearable garments, accurate placement of the electrode pairs 106, 108 may be achieved every time that the garment is worn. In this manner, consistent and reliable recordings in each therapy session may be assured. In some instances, for example, if the wearable garment 710 is a pair of shorts for stimulating the upper leg muscles, then the reference electrode 114 may be a separate sleeve 712 configured to be positioned on the calf muscle.

Next, in a step 804, the method 800 continues by positioning the wearable device 710, 712, 1000 on the muscle groups to be stimulated. In the example shown in FIGS. 12A-13D, the wearable device 710, 712 includes a pair of shorts, with the separate sleeve 712 that retains the reference electrode 114, and the muscle groups being subjected to therapy include the right and left quadriceps 716a, 716b, as well as the right and left hamstrings 718a, 718b.

Once the wearable device 710, 712 is positioned on the desired muscle groups, 716a, 716b, 718a, 718b (with an electrode pair 106 for each specified muscle group), the input device 202 (shown in FIG. 4) is powered on and a therapy cycle is selected in step 806. In one exemplary arrangement, the control system 204 and/or the server 208 may be preprogrammed with different therapy cycles that set predefined stimulation levels, as well as set stimulation and EMG measurement timing cycles. Alternatively, or in addition, the input device may be, for example, configured to allow the therapy cycle to be selectively adjusted to a desired time limit (i.e., 30 minutes), desired stimulation levels (i.e., increase stimulation), or to turn off.

Once the therapy cycle is selected in step 806, the method next initiates the selected therapy cycle in step 808. In the exemplary configuration shown in FIGS. 12A-13D, the input device is actuated to initiate a therapy cycle for two different muscle groups in each leg of a paralyzed individual. For example, the therapy cycle depicted in FIGS. 12A-12B, starts with the application of a biphasic stimulation pulse 212a, 214a, (i.e., step 810) and taking of an EMG measurement 218 of that muscle group (i.e., step 812), similar to the stimulation and EMG measurement scheme 400 shown in FIG. 8, of the right quadricep 716a. The stimulation applied is a predetermined maximum stimulation level, with the EMG measurements being taken and transmitted to the control system 204 and/or the server 208. In the example shown, after the first cycle of stimulation is applied and the EMG measurement is taken, the therapy cycle moves to the left quadricep 716b, where the stimulation and EMG cycle is repeated, i.e., the biphasic stimulation pulse 212b, 214b is applied and an EMG measurement is taken and transmitted to the control system 204 and/or the server 208. A single reference electrode 114 can be used in support of all of the EMG measurements.

In the example shown in FIGS. 13A-13B, once the left quadricep 716b has been stimulated and the EMG has been measured, the therapy cycle 806 shifts to subjecting the right hamstring 718a to the application of the biphasic stimulation and EMG measurement scheme, like the right quadricep 716a. After the right quadricep 716a is stimulated and the EMG has been measured and transmitted to the control system 204 and/or server 208, the therapy cycle 806 then shifts to the left hamstring 718b, shown in FIGS. 13C-13D. The stimulation and EMG measurement scheme is then repeated.

In one exemplary arrangement, as described above, each muscle group is subjected to a single stimulation cycle and EMG measurement action before moving to the next muscle group. Once all the selected muscle groups have undergone a stimulation and EMG measurement action, the process will repeat itself until the therapy cycle reaches its predetermined time limit. However, it is understood that alternative therapy cycles 808 are also contemplated.

For example, as described in connection with FIG. 7, if channel 402 represents the right quadricep 716a muscle group, and channels 404, 406 and 408 represent the left quadricep 716b, right hamstring 718a, left hamstring 718b, respectively, then the right quadricep 716a is subject to stimulation, followed shortly by either a synchronized or staggered stimulation of the left quadricep 716b, right hamstring 718a, and left hamstring 718b. EMG measurements 218 are taken of the right quadricep 716a between stimulation pulses for a set time period during the therapy cycle, and the EMG measurements 218 of the right quadricep 714a are aggregated in a memory unit of the control system 204 or transmitted to the server 208. After the set time period expires, EMG measurements are then taken of the next muscle group, i.e., the left quadricep 716a, and aggregated in the memory unit of the control system 204 or transmitted to the server 208, until a second set time period expires. The same process is repeated for the right and left hamstring 718a, 718b.

In yet a further alternative arrangement, each individual muscle group may undergo a full therapy cycle of stimulation, EMG measurement, and transmission of the EMG measurements to the memory of the control system 204 and/or the server 208 before moving to a subsequent, individual muscle group, until all the muscle groups are completed.

Regardless of which therapy cycle 808 approach is taken, once the EMG measurements are taken by the electrode pairs, the EMG measurements are transmitted in step 814 to either memory of the control system 204 or the server 208, where the EMG measurements are aggregated for each muscle group, and in step 816, are used to determine an average peak EMG measurement for each therapy session, as previously discussed above in connection with FIGS. 10A-10C. Such data is particularly useful for completely paralyzed individuals to determine the degree of atrophy, if any. In a step 818, the results of the peak EMG measurements are transmitted to a display device, such as a monitor or a handheld device. The results are then used to adjust the therapy cycle in step 820.

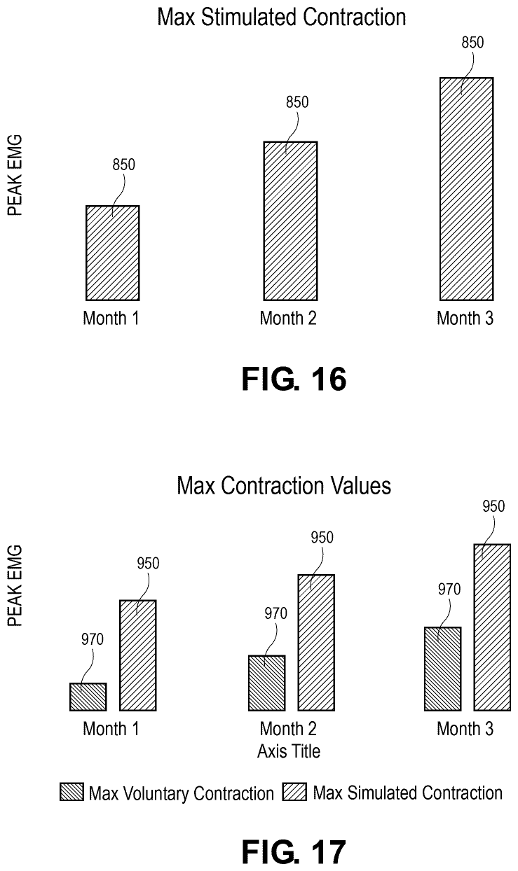

If therapy is still needed (step 822), the method 800 may be repeated for several months to track the progress of the muscle group's response to the stimulation therapy. For example, FIG. 16 illustrates three months of data utilizing method 800. As may be seen, a maximum stimulated contraction 850 that can be achieved with method 800 increases progressively month over month, due to the repeated application of the stimulation therapy.

For individuals that are partially paralyzed, similar methods may be employed for applying stimulation and taking EMG measurements as discussed in connection with FIGS. 12A-13D and FIG. 14. However, partially paralyzed individuals will still have some voluntary control over muscles. As discussed above in connection with FIG. 10A-10C, tracking peak values for EMG signals for such manual contractions can be used to generate trend lines to assist in the determination of treatment plans to utilize, or to determine if treatment is working. Accordingly, referring to FIG. 15, a method 900 of operating an exemplary configuration of the wearable stimulation and EMG measurement arrangement 100 on a partially paralyzed individual 700 will now be discussed.

Similar to the method 800 set out in FIG. 14, the method 900 begins in step 902 by providing one or more wearable devices. For ease of explanation, the example of utilizing a pair of therapy shorts for treating the quadriceps 716a, 716b, and hamstrings 718a, 718b of the right and left legs will be utilized for explanation of the present method. The wearable device is positioned on the desired muscle groups in step 904.

Once positioned, the input device is powered up and utilized to capture EMG measurements for voluntary contractions of each muscle group in step 906. For example, for each muscle group, the control system 204 activates the EMG measurement function of a set electrode pair, for example, a pair associated for with right quadricep 716a. Once activated, in the step 908, the input device prompts the user to maximally contract the right quadricep 716a and the EMG measurement device 120 is activated to measure EMG. Once the measurement is taken, the data is sent to the memory in the control system 204 and/or the server 208 and stored in step 910. Steps 908 and 910 are repeated a predetermined number of times, if a threshold number has not been reached (step 911). In one exemplary arrangement, steps 908 and 910 are repeated three times. Once all of the needed EMG measurements are taken, in step 912, the separate EMG measurements are averaged to determine a maximum voluntary contraction level.

Using the maximum voluntary contraction level information, a maximum stimulus level is then determined in step 914. Using the predetermined maximum stimulus level from step 914, the stimulator device 122 control system 204 applies the predetermined maximum stimulus to the targeted muscle group in step 916, i.e., the right quadricep 716a in this example. As shown in FIGS. 6A, 6B, the stimulus in step 916 is applied in a biphasic manner. Between application of a cycle of stimulation pulses, 212, 214, an EMG measurement 218 is taken in step 918. Data from the EMG measurements 218 are transmitted to the memory in the control system 204 and/or the server 208 and stored in step 920. Once all of the EMG measurements 218 are taken, in step 922, the separate EMG measurements are averaged to determine a maximum stimulated contraction level for that therapy event.

The method 900 may be repeated for several months if needed (step 924) to track the progress of the muscle group's response to the stimulation therapy. For example, FIG. 17 illustrates three months of data utilizing method 900. As may be seen, a maximum stimulated contraction 950 that can be achieved with method 900 is significantly higher than a maximum voluntary contraction 970 that can be achieved. Over time, however, the maximum voluntary contraction 970 increases progressively, as the maximum stimulation contraction 950 increases, due to the application of the stimulation.

The method 900 may be repeated for each individual muscle group. In one exemplary arrangement, the voluntary contractions performed in step 906 and EMG measurements taken in step 908 are undertaken for multiple muscle groups. For example, in the exemplary arrangement of FIGS. 12A-13D, steps 906 and 908 are undertaken for the first muscle group, i.e., the right quadricep 716a. Then steps 906 and 908 are repeated for the second muscle group, i.e., the left quadricep 716b. The steps may be further repeated for the right and left hamstring muscles 718a, 718b. Once these steps are taken, then the predetermine maximum stimulus may be applied to each group, i.e., steps 916-920. In one exemplary arrangement all of the muscle groups may undergo the stimulation activity with the control system controlling the stimulator software to rotate from one muscle to the next. Alternatively, the entire method 900 may be performed for each targeted muscle group, and then the method 900 may be repeated for each successive muscle group until each muscle group is completed.

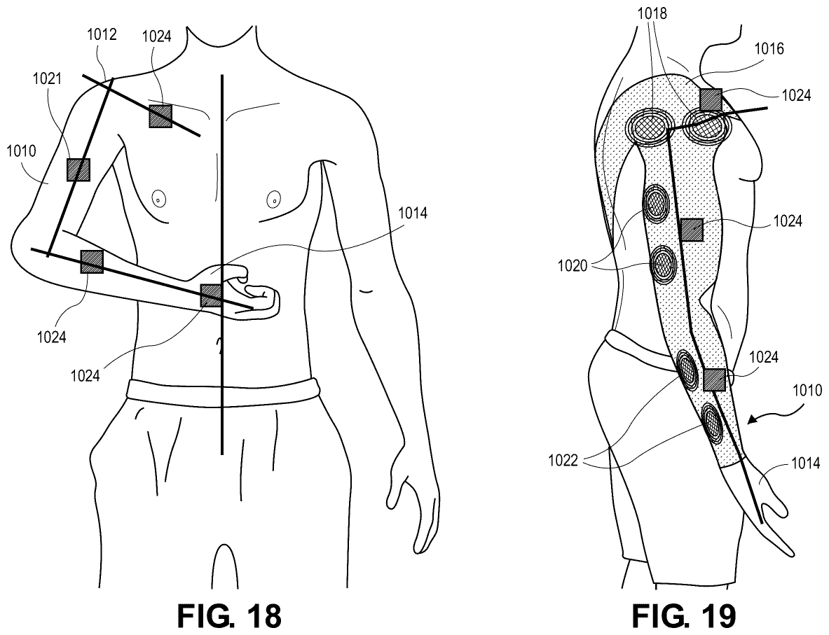

Referring to FIGS. 18-21C, an exemplary arrangement of a wearable device 1016 that may be utilized to provide stimulation therapy according to either of the methods 800 or 900 for paralysis or injury to an arm 1010 and/or shoulder 1012 is shown. For example, certain individuals experience hemiplegia, a one-sided paralysis as a result of brain or spinal cord injuries. In such individuals, arm position is highly dependent upon hypertonicity of the biceps, wrists, and finger flexors. Typically, hypertonicity tends to induce flexion at the elbow, such that arm posturing presents as shown in FIG. 18. The arm 1010 is bent to an approximately 90° angle with the wrist or hand 1014 being directed toward a center line of the individual. Increasing muscle strength and arm extension encourages arm usage and reduces the effects of hemiplegia/hemiparesis. Accordingly, muscle stimulation can counter the angular forces that cause involuntary bicep and finger flexor contractions and a reduced range of motion.

In one exemplary arrangement, referring specifically to FIG. 19, and 21A-21C, a wearable sleeve 1016 may be provided that includes at least one electrode pair that is connected to an input device 1017. In the arrangement depicted in FIG. 19, and 21A-21C, there are three sets of electrode pairs, a shoulder electrode pair 1018, a triceps electrode pair 1020, and a finger extensor electrode pair 1022. Like the arrangement 100, the electrode pairs 1018, 1020, and 1022 are all configured to deliver stimulation, as well as to take EMG measurements. A reference electrode may be provided, for example, at a location on a back or chest of the user. The shoulder electrode pair 1018 provides stimulation to the shoulder muscles and contributes to an individual's ability to raise the subject arm 1010 up. The triceps electrode pair 1020 stimulates the triceps which allows for the arm 1010 to extend at the elbow. Finally, the finger extensor electrode pair 1022 stimulates the finger extensor muscles and allows the fingers to extend or uncurl.

In addition to the electrode pairs 1018, 1020, and 1022, the wearable sleeve 1016 further comprises a plurality of Inertial movement units, i.e., IMUs 1024. The IMUs 1024 are integrated into the wearable sleeve 1016 similar to the electrode pairs 1018, 1020, 1022. The IMUs 1024 are operatively connected to the control system 204 and data collected by the IMUs 1024 may be transmitted to the control system 204 and/or the server 208. More specifically, the IMUs are useful to track different positions of each joint, as well as monitor those positions over time to be able to selectively adjust therapy schemes for an individual. For example, the IMUs can be used to determine the position of the shoulders, elbows, wrist, and fingers. These positional measurements assist a caregiver to determine a level of arm usage, as well as a velocity of the movement.

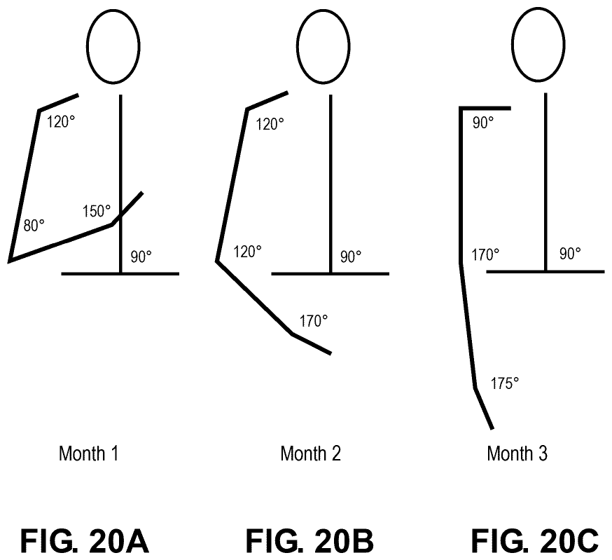

A method of operating the wearable sleeve 1016 may largely follow the method 900 set forth in FIG. 15. However, instead of tracking voluntary contractions, or in addition to tracking the voluntary contraction in steps 906, 908 and 910, IMU measurements may be taken and stored. The method 900 may be repeated for several months to track the progress of the muscle groups' response to the stimulation therapy. For example, FIGS. 20A-20C illustrates three months of positional data utilizing method 900. As may be seen, the shoulder muscle 1012 may only allow an arm 1010 to move outward 120° from the shoulder 1012, in the first two months, but in the third month after application of stimulation, may be able to fully move the shoulder to a 90° angle. When looking at the elbow flexion in month one, only an 80° angle may be achieved, whereas by the second month, an angle of 120° flexion is possible. And in the third month, a flexion of 170° is possible. The wrist 1014 may only allow the fingers to achieve 150° flexion, in the first month, while in the second month 170° flexion can be achieved, up to 175° flexion by the third month.

One of the advantages of the above-described wearable garments 1016 is that the therapy can be applied at an individual's home and on a schedule that is convenient for the individual, thereby increasing therapy opportunity. In addition, the electrodes can be accurately placed every time, as the garment 1016 will provide repeatability in the positioning of the electrodes, without requiring a professional to apply the electrodes each time therapy is needed.

What have been described above are examples of the present disclosure. It is, of course, not possible to describe every conceivable combination of components or methodologies for purposes of describing the present invention, but one of ordinary skill in the art will recognize that many further combinations and permutations of the present invention are possible. While certain novel features of this disclosure shown and described below are pointed out in the annexed claims, the disclosure is not intended to be limited to the details specified, since a person of ordinary skill in the relevant art will understand that various omissions, modifications, substitutions and changes in the forms and details of the disclosure illustrated and in its operation may be made without departing in any way from the spirit of the present disclosure. Accordingly, the present disclosure is intended to embrace all such alterations, modifications, and variations that fall within the scope of the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements. No feature of the disclosure is critical or essential unless it is expressly stated as being “critical” or “essential.”

What is claimed is:

1. An electrical stimulator device, comprising:
a first pair of electrodes configured to be applied to a first target muscle group of a user;
a second pair of electrodes configured to applied to a second target muscle group;
a reference electrode configured to be applied to a location on a body of the user;
a control system having switch circuitry;
a stimulation device; and
an EMG measurement device;
wherein the control system switch circuitry operatively connects the stimulation device and the EMG measurement device to the first pair of electrodes and to the reference electrode such that when the stimulation device is activated, the first electrode pair of electrodes is operative to stimulate the first target muscle group.

2. The electrical stimulator device of claim 1, wherein the control system switch circuity is configured to turn the stimulation device off from the first pair of electrodes and turn stimulation device on for the second pair of electrodes.

3. The electrical stimulator device as claimed in claim 1, wherein the control system switch circuity is configured to turn the stimulation device off for all electrode pairs and turn on the EMG measurement device for one of the first and second pair of electrodes, and the reference electrode.

4. The electrical stimulator device as claimed in claim 1, further comprising an input device that is operatively connected to the control system.

5. The electrical stimulator device as claimed in claim 1, further comprising a server operatively connected to the control system, the server having a memory to store EMG measurements and provide computational analysis of the EMG measurements.

6. The electrical stimulator device as claimed in claim 5, further comprising one or more inertial movement units operatively connected to the control system, wherein the one or more inertial movement units are configured to track movement and position of the first or second targeted muscle group.

7. The electrical stimulator device as claimed in claim 6, wherein the electrical stimulator device is a wearable garment.

8. A method of applying a stimulation therapy, comprising:
positioning an electrical stimulator device such that a first set of electrodes are positioned across a first target muscle group of a user and a second set of electrodes are positioned across a second target muscle of the user; and a reference electrode is position on a location of a body of the user;
selecting one or more stimulation parameters for a stimulation cycle;
initiating a stimulation cycle for the first target muscle group by providing a biphasic pulse followed by an EMG measurement collection and repeating application of the biphasic pulse followed by the EMG measurement collection for a predetermined time period; and
aggregating data from the EMG measurement collection to demonstrate a response to the biphasic pulse of the stimulation cycle by the first target muscle group.

9. The method of claim 8, wherein after the stimulation cycle is performed for the first target muscle group, a second stimulation cycle is performed for the second target muscle group, by providing biphasic pulse followed by EMG measurement collection for a second predetermined time period, and aggregating data from the EMG measurement collection for the second predetermined time period to demonstrate a response to the bephasic pulse of the second stimulation cycle by the second target muscle group.

10. The method of claim 8, further comprising initiating voluntary contractions of the first target muscle group, and taking EMG measurements of the voluntary contractions for a predetermined number of times, recording the EMG measurements of the voluntary contractions, and comparing the EMG measurements of the voluntary contractions with the EMG measurements collected after stimulation to make a determination on adjusting therapy.

11. The method of claim 8, further comprising tracking positional data of the first target muscle group over a predetermined time period.

12. The method of claim 8, wherein after the stimulation cycle is applied to the first target muscle group, a second stimulation cycle is initiated for the second target muscle group by providing a biphasic pulse followed by an EMG measurement collection and repeating application of the biphasic pulse followed by the EMG measurement collection for a second predetermined time period.

13. The method of claim 8; wherein after the stimulation cycle is initiated for the first target muscle group, a second stimulation cycle is imitated for the second target muscle group, and wherein an EMG measurement collection is performed during a common gap between each biphasic pulse of each stimulation cycle.

14. The method of claim 13, wherein a third electrode pair is positioned across a third target muscle group, and wherein a stimulation cycle is initiated for each of the first, second and third target muscle groups.

15. The method of claim 14, wherein the stimulation cycle for the second target muscle group is delayed until after more than one biphasic pulses is applied to the first target muscle group, and wherein the stimulation cycle for the third target muscle group is delayed until after more than one biphasic pulses is applied to the second target muscle group, and wherein an EMG measurement collection is performed during a common gap between each biphasic pulse of the more than one biphasic pulses of the first, second and third muscle groups.
